(19) United States
(12) Patent Application Publication
(10) Pub. No.: US 2007/0079418 A1
(43) Pub. Date: Apr. 12, 2007

(54) Headband

(76) Inventor: Lajos Fehervizy, Mosonmagyarovar (HU)

Correspondence Address: VENABLE LLP, P.O. BOX 34385, WASHINGTON, DC, 20043-9998, US

(21) Appl. No.: 10/577,015

(22) PCT Filed: Jul. 27, 2004
(86) PCT No.: PCT/HU04/00081

§ 371 Date: Apr. 25, 2006

(30) Foreign Application Priority Data
Nov. 4, 2003 (HU) U0300268

(51) Int. Cl. A61F 9/06

(52) U.S. Cl. 2/8.3

(57) ABSTRACT

The invention relates to a headband for protection of the head of small children, having a band body (1) consisting of at least one fore-arc (2) and a backside arc (3), and the at least one fore-arc (2) has a central portion (10) arranged above the nose, and a protecting band (24) is attached to the backside arc (3) to protect the face of the user.

### FIELD OF THE INVENTION

The invention relates to a protective headband for protection of the head of a toddler, having a rounded band body consisting of at least one fore-arc and a backside arc, said fore-arc has a central portion located above the nose in the vertical symmetry line of the body, backside arc is attached to the at least one fore-arc in connecting areas, and the width of the backside arc is greater than the maximum width of the fore-arc, and the arc length of the backside arc is shorter than the total arc length of the at least one fore-arc, and the distance measured between the lower rim of the backside arc and the lower rim of the fore-arc is smaller than the width of the backside arc.

### BACKGROUND ART

It happens again and again in the flat, garden, public institutions, i.e. reception room or playground, that a toddler losing his/her balance has a fall and bumps his/her head against some hard object, i.e. on the floor, cupboard, the edge of a sandpile or a door-jamb. Due to the bump the toddler can suffer a severe or slight injury on the head, in most cases just on the brow or the nape and last but not least on the face.

To solve this daily problem several resolutions are provided. The patent description No. U.S. Pat. No. 4,581,773 discloses a protective hatgear for infants and young children, which comprises a head-enveloping member having the appearance of a conventional hat, but constructed of a core of impact absorbent foam, encapsulated within a shell of textile fabric material, the textile fabric material providing the inner and outer surfaces of the garment.

The granted patent U.S. Pat. No. 5,343,569 discloses a protective helmet, particularly used by children, which is provided to include an enclosed, generally cylindrical helmet structure providing a plurality of layers of first and second density polymeric foam respectively, with a chin panel mounted to a first side of the helmet arranged for securement to a second side of the helmet.

From the European Patent No. EP 0 638 249 a protective hat can be known for children and adults, which comprises a head-enveloping member constructed of a core of impact absorbent foam, encapsulated within a shell of textile fabric material, the textile fabric material providing the inner and outer surfaces of the hat. The core is so constructed as to provide for conformity to the shape of head of the wearer, and to allow for ventilation.

The main disadvantage of above referenced resolutions is that wearing thereof is especially uncomfortable both in summer or in a hot room, since they cover all the skull and are secured by a chin-rested band, moreover, these helmets do not protect the face from injuries.

To overcome the discommodities mentioned above the U.S. Pat. No. 4,745,637 provides a head protector for children and invalids, which has a series of concentric rings of tubular fabric packed with yieldable material and a top member of yieldable material, and straps binding said rings and top together, a ring of spaced cushions, or pillows, between the lowermost ring and the adjacent ring, and means for positioning said protector on a user's head and maintaining same against rearward movement.

A protective head cover according to the European Patent EP 0 286 726 consists of a head ring, extending over the forehead, temples and back of the head, and of two crossover safety straps extending over the head. In order to improve a protective head cover of this type and, in particular, to achieve a protection of the fontanel which is at particular risk in babies and small children, one of the safety straps extends from one ear to the other and the other safety strap extends from the forehead to the back of the head in order to protect the skull and the fontanel.

For example, the head protector according to the patent U.S. Pat. No. 4,745,637 provides protection only in the line of the forehead and above.

Protection of toddlers' head against injuries occurring in the face and the nape by a comfortable way were therefore unsolved.

However, this problem may virtually be solved by the resolution proposed in the patent description HU 2355 U disclosing a headband for protection of the head of a child against injuries caused by bumps. This headband consists of a band body having a fore-arc and a backside arc, and the fore-arc has an extending central portion arranged above the nose. This arrangement is really light and airy and protecting effectively the most critical areas of the head against injuries because of its structural design, despite of having no chin-rested secure belt. Nevertheless, it has a disadvantage, namely has no effective protective ability against injuries endangering the face.

### THE OBJECT OF THE INVENTION

Therefore, the main object of the present invention is to provide an improved head protection device for toddlers against injuries caused by bumps, which on one hand protects effectively the parts of the head most sensible in relation of injuries, especially the forehead as well as the nose, the temple and the backside of the head, on the other hand it also provides effective protection against injuries endangering the mouth and the chin.

### DISCLOSURE OF THE INVENTION

The object mentioned above is achieved by developing a headband according to the present invention, for protection of the head of a toddler, having a rounded band body consisting of at least one fore-arc and a backside arc, said fore-arc has a central portion located above the nose in the vertical symmetry line of the body, backside arc is attached to the at least one fore-arc in connecting areas, and the width of the backside arc is greater than the maximum width of the fore-arc, and the arc length of the backside arc is shorter than the total arc length of the at least one fore-arc, and the distance measured between the lower rim of the backside arc and the lower rim of the fore-arc is smaller than the width of the backside arc, and the a face-protecting band consisting of a right side stem and a left side stem and a face protecting member fitted therebetween is connected to the backside arc, said right side stem is fitted to the right side of the backside arc, and the left side stem is fitted to the left side of the backside arc

Said right side stem is advantageously fitted to the right side of the backside arc, and the left side stem is fitted to the left side of the backside arc by means of dead joints respectively.

The width of the face protecting member is suitably greater than the width of the stems.

Said face-protecting band is favourably formed in at least one layer made of an energy absorbing material.

The face-protecting band is advantageously formed with several layers, so that an external surface of an energy absorbing layer is provided by a harder protecting layer.

Said harder protecting layer is suitably made of impact-resistant plastic material.

The headband according to the present invention will be disclosed in details by way of its most feasible embodiment based on the accompanying drawing attached. In the drawing

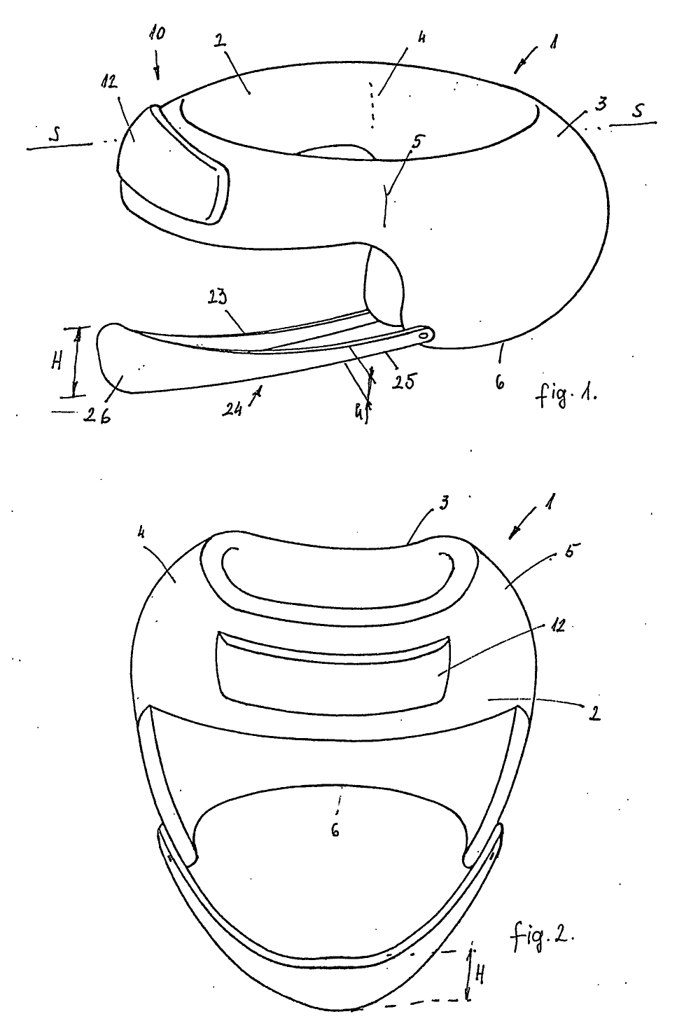

FIG. 1 is a side perspective view of a headband according to the present invention,

FIG. 2 is a front perspective view of a headband according to the present invention.

### DETAILED DESCRIPTION OF THE INVENTION

As it can be shown in the FIG. 1. an advantageous embodiment of the headband according to the present invention is provided with a rounded body 1 divided into two arcs 2, 3 having different sizes and accurately fitting to the curve of the head. The fore-arc 2 and the backside arc 3 are attached to each other in connecting areas 4, 5. This two connecting areas 4, 5 are located just behind the ears and a little bit above thereof. The fore-arc 2 serves as a protective means to protect the forehead and the temple above the ears and the brow. The width of the fore-arc 2 is adequately sized to cover the greater part of the forehead between the brow and the scalp. The width of the backside arc 3 is greater than the maximum width of the fore-arc 2, so that it covers the backside of the head between the ears as well as between the upper contour line of the body 1 and the lower contour line of the nape, therefore it serves as a protective means to protect the nape, because its lower rim 6 is located along the lower contour line of the same. The fore-arc 2 has a central portion 10 located above the nose provided with a face protection bulge 12 arranged in order to partly protect the nose in case of falling down to face.

It is very clear from the FIG. 1. that a face-protecting band 24 is connected to the body 1. This face-protecting band 24 consists of narrower stems 23, 25, that is a right side stem 23 and a left side stem 25, and a face protecting member 26. The right side stem 23 is fitted to a first side of the lower rim 6 of the backside arc 3, and a left side stem 25 is fitted to a second side of the lower rim 6.

The body 1 of the headband of the present invention is advantageously formed in one closed piece, or with a fore-arc 2 divided into two part by a central slot, as it can be known from the document HU 2355 U mentioned above.

FIG. 2. is a front perspective view of a further embodiment of the headband according to the present invention, where the rounded body 1 consist of at least one fore-arc 2 and a backside arc 3, and the at least one fore-arc is provided with a face protection bulge 12 arranged on its central portion 10 located above the nose in the vertical symmetry line of the body 1. The backside arc 3 is attached to the at least one fore arc at the connecting areas 4, 5, and the width of the backside arc 3 is greater than the width of the at least one fore-arc 2. It can be seen, that the arc length of the backside arc 3 is shorter than the total arc length of the at least one fore-arc 2. The distance measured between the lower rim 6 of the backside arc 3 and the lower rim of the fore-arc 2 is smaller than the width of the backside arc 3. The face protecting band 24 having a right side stem 23 and a left side stem 25 and a face protecting member 26 is connected to the backside arc 3, and the right side stem 23 is fitted to the right side of the backside arc 3, further the left side stem 25 is fitted to the left side of the backside arc 3. This connection is advantageously a fixed type fastening, since the face protecting member 26 fulfil its function optimally if it is not movable relative to the body 1.

It is clearly seen in both FIGS. 1. and 2. that a width H of the face protecting member 26 is greater than the width of the stems 23, 25 in order to protect a greater area of the face located around the mouth.

Although it is not depicted in the figures, in a more advantageous embodiment the face-protecting band 24 may be formed in at least one layer 7 made of an energy absorbing material being adequately resilient to absorb the most part of the energy in case of falling down to face. Nevertheless, the face-protecting band 24 might be formed with several layers, so that an external surface of an energy absorbing layer 7 (i.e. foam rubber or the like) is provided by a harder protecting (i.e. plastic) layer 8. By this arrangement the durability of the whole headband, especially the face protecting member 26 may be increased. In this case the harder protecting layer 8 may be made of impact-resistant plastic material.

The headband according to the invention has several advantages, therefore being adapted to serve as a protective device for protection of head of a toddler or other physically unbalanced users (like persons having illnesses accompanied by imbalance) against injuries caused by bumps. The headband according to the invention effectively protects not only the forehead, the nose, the temple and the nape, but also the face with highly comfortable wearing features. Due to its light structure—in spite of serving effective protection—it does not confine movement of the user, it does not restrict his/her visual area and audition, it is easily applicable as well as removable, and its fitting does not require extended procedure. Additionally, it can be produced easily at a low cost.

1. Headband for protection of the head of a toddler, having a rounded band body (1) consisting of at least one fore-arc (2) and a backside arc (3), said fore-arc (2) has a central portion (10) located above the nose in the vertical symmetry line of the body (1), backside arc (3) is attached to the at least one fore-arc (2) in connecting areas (4, 5), and the width of the backside arc (3) is greater than the maximum width of the fore-arc (2), and the arc length of the backside arc (3) is shorter than the total arc length of the at least one fore-arc (2), and the distance measured between the lower rim (6) of the backside arc (3) and the lower rim of the fore-arc (2) is smaller than the width of the backside arc (3), characterised in that the a face-protecting band (24) consisting of a right side stem (23) and a left side stem (25) and a face protecting member (26) fitted therebetween is connected to the backside arc (3), said right side stem (23) is fitted to the right side of the backside arc (3), and the left side stem (25) is fitted to the left side of the backside arc (3).

2. Headband according to the claim 1, characterised in that said right side stem (23) is fitted to the right side of the backside arc (3), and the left side stem (25) is fitted to the left side of the backside arc (3) by means of dead joints respectively.

3. Headband according to the claim 2, characterised in that a width (H) of the face protecting member (26) is greater than the width (h) of the stems (23, 25).

4. Headband according to the claim 3, characterised in that said face-protecting band (24) is formed in at least one layer (7) made of an energy absorbing material.

5. Headband according to the claim 3, characterised in that the face-protecting band (24) is formed with several layers (7, 8), so that an external surface of an energy absorbing layer (7) is provided by a harder protecting layer (8).

6. Headband according to the claim 5, characterised in that said harder protecting layer (8) is made of impact-resistant plastic material.
